(19) United States
(12) Patent Application Publication
(10) Pub. No.: US 2004/0024236 A1
(43) Pub. Date: Feb. 5, 2004

(54) (Stabilised) peroxide compositions phlegmatised with a specific unsaturated phlegmatising agent

(76) Inventors: Koen Antoon Kornelis Vanduffel, Deventer (NL); Marinus Catharinus Tammer, Diepenveen (NL); Petrus Paulus Waanders, Goor (NL); Andreas Petrus Van Swieten, Velp (NL)

Correspondence Address: Richard P. Fennelly, Akzo Nobel Inc., Intellectual Property Dept., 7 Livingstone Avenue, Dobbs Ferry, NY, 10522, US

(21) Appl. No.: 10/462,950

(22) Filed: Jun. 17, 2003

Related U.S. Application Data
(60) Provisional application No. 60/390,799, filed on Jun. 20, 2002.

(51) Int. Cl. C08F 4/32; C07C 69/96

(52) U.S. Cl. 558/264; 526/230.5

(57) ABSTRACT

The invention relates to peroxide compositions that are phlegmatised with a specific unsaturated phlegmatising agent. The phlegmatised peroxide compositions are optionally stabilised. In addition, the invention relates to the safe handling of these peroxide compositions and the use of these peroxide compositions in polymerisation processes.

The invention relates to phlegmatised peroxide compositions that are optionally stabilised, that can be handled, produced, and shipped in a safe manner. It also relates to the safe use of such peroxides compositions in polymerisation processes where the resulting polymer preferably has a reduced level of undesired residues of low-molecular weight and/or inert phlegmatising agents.

Stabilised peroxide compositions, such as peroxydicarbonate compositions, have been disclosed before:

In the Journal of the American Chemical Society, Volume 72, pp. 1254-1263 (1950) it is mentioned that the decomposition of diisopropyl peroxy-dicarbonate is retarded by the addition thereto of substances such as iodine, phenol, hydroquinone, salicylic acid, nitromethane, pyrogallol, cyclohexene, or hydrogen peroxide (HOOH).

U.S. Pat. No. 5,155,192 discloses the use of organic hydroperoxides (ROOH, wherein R represents an organic group) for the stabilisation of peroxydicarbonates.

U.S. Pat. No. 5,892,090-A1 describes the stabilisation of peroxydicarbonates against decomposition by the presence of an effective amount of one or more oximes.

JP 10,059,933-A discloses that decomposition of peroxydicarbonates can be retarded with beta-dicarbonyl, or cyclic alpha-diketone compounds.

JP 10,059,932-A describes the stabilisation of peroxydicarbonate by using phosphomolybdic acid.

Due to the safety hazards associated with most (organic) peroxides, they are often diluted with one or more specific solvents, also known as phlegmatisers. Classical phlegmatising agents are hydrocarbons and esters, such as phthalates. The use of phlegmatising agents for (organic) peroxides has been disclosed before:

U.S. Pat. No. 4,131,728 discloses a polymerisation process employing shock-sensitive peroxides in improved phlegmatisers. The improved phlegmatisers are specific monomers that do not homopolymerise. Exemplified suitable phlegmatising monomers are maleic and citraconic anhydride and esters thereof, fumarates and fumaronitriles, cinnamates and cinnamonitriles, and stilbene.

U.S. Pat. No. 4,029,875 discloses an ethylene polymerisation process employing a mixture of organic peroxides and cyclic alkenes, styrene, or styrene homologues bearing alkyl substituents on the benzene nucleus to reduce the consumption of initiator in the process and to improve the optical and mechanical properties of the polyethylene produced.

However, conventional phlegmatisers and stabilisers were often observed to adversely effect the polymerisation process in which the peroxide composition was used. Accordingly, there is a need in the industry for packaged stabilised peroxide formulations that can be produced, handled, and shipped in a safe manner and where the stabilised peroxide formulations can safely be used in polymerisation reactions without any adverse effect. Preferably, use is made of specific stabilised peroxide formulations that lead to a reduction in the amount of undesired phlegmatising agent in the polymer (resin) that is produced with said formulations. Conventional phlegmatising agents are not suitable for this function.

Surprisingly, it has now been found that the use of a specific phlegmatising agent in a (stabilised) peroxide composition results in a final composition that shows good stability, milder effects in the decomposition of the peroxide, and no adverse effect in the polymerisation process. In addition, polymers, e.g. PVC, prepared using a peroxide composition of the invention, show unexpected properties regarding morphology, processing and stability. The use of an olefin as phlegmatising agent has such a positive influence on the decomposition behaviour of the peroxide composition that, depending on the circumstances, it can be handled, produced, and shipped at higher temperatures than a conventional stabilised peroxydicarbonate composition. In addition, when a (stabilised and) phlegmatised peroxide composition of the present invention is used in a polymerisation reaction, the phlegmatiser of the invention preferably is “consumed” (i.e. the olefin is a reactive phlegmatiser) during the polymerisation reaction, which gives the benefit of reduced unbound phlegmatiser in the polymer (so that the polymer contains less volatile product). The reduced unbound phlegmatiser levels improve the organoleptic properties of the resulting (co)polymer and may even obviate a treatment of the polymer to reduce volatile material.

Accordingly, we claim 1) specific peroxide compositions that comprise i) optionally a stabiliser; and ii) an specific agent as phlegmatising agent; 2) the production, handling, and shipping of such peroxide compositions which is more safe; and 3) the use of these peroxide compositions in polymerisation processes.

The peroxides that can be used for the compositions according to the invention are specific well-known compounds of which many are commercially available. The peroxides are preferably liquid at the temperature at which they are produced, handled, or shipped. If the peroxides are not liquid they may be dissolved in a solvent, or a mixture of solvents. The peroxides are used as initiators in free radical polymerisation processes, and they are of the structural formulae:

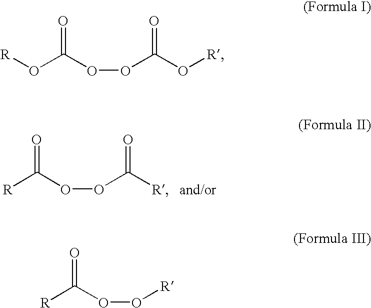

wherein R and R′ represent organic groups. R and R′ generally each have I to 20 carbon atoms, preferably 2 to 18 carbon atoms, and more preferably 2 to 16 carbon atoms. Preferably, R and R′ represent branched or non-branched, substituted or unsubstituted alkyl, alkenyl or cycloalkyl groups. As suitable substituents may be mentioned aromatic groups, halogen atoms, such as chlorine and bromine, nitro groups, aryloxy groups and alkoxy groups. As examples of R and R′ may be mentioned methyl, ethyl, n-propyl, isopropyl, nbutyl, sec-butyl, tert-butyl, isobutyl, hexyl, octyl, 2-ethylhexyl, 1,1 dimethylpropyl, 1,1,3,3 tertramethylbutyl, 1,1 dimethyl-3-hydroxybutyl, lauryl, mirystyl, cetyl, stearyl, allyl, methallyl, crotyl, cyclohexyl, 4-tert-butylcyclohexyl, 4-tertamylcyclohexyl, benzyl, 2-phenylethyl, 2-phenylbutyl, 2-phenoxyethyl, 2methoxyethyl, 2-ethoxyethyl, and 3-methoxybutyl. Although R and R′ are generally identical for the peroxides according to Formula I and Formula II, the invention is not limited to these symmetric peroxides. Specifically for products of Formula III, R preferably is selected such that products of the formula RCOOH are conventional acids such as neodecanoic, neononanoic, neooctanoic, neoheptanoic, and 2-ethyl-hexanoic acid.

For example, in the case of peroxydicarbonates of the Formula I, also asymmetric peroxydicarbonates such as isopropyl-sec-butyl-peroxydicarbonate, mixtures of asymmetric peroxydicarbonates, and mixtures of symmetric and asymmetric peroxydicarbonates, such as the mixtures of diisopropyl peroxydicarbonate, di(sec-butyl) peroxydicarbonate and isopropyl-(sec-butyl) peroxydicarbonate as described in U.S. Pat. No. 4,269,726 can be stabilised and phlegmatised according to the present invention. The peroxide compositions according to the invention preferably contain at least one peroxide of the Formula I and/or Formula II. Most preferably the peroxide compositions according to the invention preferably comprise at least a peroxide according to formula I.

The stabiliser that is used according to the invention is any conventionally used stabiliser. The preferred stabiliser is a hydroperoxide, the most preferred stabiliser is tert-butyl hydroperoxide.

The specific compounds that are used as phlegmatiser, and preferably being reactive, are of the general formula R″HC═CHR′″ (Formula IV), wherein R″ and R′″ are independently selected from hydrogen and the group consisting of linear or branched, saturated or unsaturated C₁-C₁₂ alkane moieties, and R″ and R′″ may be connected to form a cyclic alkene, with the exception of cyclohexene. Preferably, R″ and R′″ are such that no conjugated double bonds are present, such as in 1,3-dienes. Preferred phlegmatisers according to Formula IV are selected from:

the group of a-olefins consisting of 1-hexene, 1-heptene, 1-octene, 1-nonene, 1-decene, 1-undecene, and 1-dodecene,

the group of cyclic alkenes consisting of cycloheptene, cyclooctene, and cyclododecene, and

mixtures of any of the preferred phlegmatisers.

The use of α-olefins is more preferred. The most preferred α-olefinic phlegmatiser is 1-octene.

The phlegmatiser used in accordance with the present invention preferably reacts efficiently in the polymerisation process employing the peroxide composition. The term “react efficiently” as used herein means that at least 25%, preferably at least 50%, and most preferably more than 75% by weight of the reactive phlegmatiser is reacted in the polymerisation process. In other words, in the most preferred situation, less than 25% by weight of the phlegmatiser used in the process is extractable from the resin (without destruction of the resin). Since the peroxide compositions according to the invention are pre-eminently suited for use in conventional suspension vinyl chloride polymerisation processes, it is preferred that the phlegmatiser reacts at the conditions of said conventional processes, which are typically conducted at temperatures of 40-80° C. and pressures of up to 18 bara. Whether or not it is a reactive phlegmatiser is easily tested by checking the amount of unreacted phlegmatiser in the polymer. It is to be understood that the term “reactive phlegmatiser” does not relate to conventional phlegmatisers, which do not react and often plasticise the resulting resin.

Phlegmatised peroxide compositions, according to a preferred embodiment of the invention, comprise:

from 20 to 95% by weight, based on the weight of the total composition (% w/w), of at least one peroxide of Formula I, optionally in combination with at least one peroxide of the Formula II and/or III. Preferred peroxides are peroxydicarbonates (of Formula I) optionally combined with diacylperoxides (of Formula II). More preferred are just peroxydicarbonates. Even more preferred are liquid peroxydicarbonates. Most preferred are di(2-ethylhexyl) peroxydicarbonate, di(n-butyl) peroxydicarbonate, and di(sec-butyl) peroxy-dicarbonate,

from 0 to 1% w/w of stabiliser. Preferably the amount of stabiliser is from 0.05 to 0.5% w/w, and most preferably from 0.1 to 0.3% w/w. The preferred stabiliser is a hydroperoxide, the most preferred stabiliser is tert-butyl hydroperoxide,

from 5 to 90% w/w of phlegmatising agent according to Formula IV, more preferably this amount is from 10 to 75% w/w, and most preferably from 15 to 60% w/w. Preferred phlegmatising agents are selected from the group of cyclo alkenes (with the exception of cyclohexene), and/or olefins. More preferred phlegmatiser is an α-olefin. The most preferred α-olefinic phlegmatiser is 1-octene, and

from 0 to 50% w/w of optional conventional phlegmatisers, up to a total of 100% .

In another preferred embodiment, the invention relates to phlegmatised peroxide compositions wherein the selection of peroxide(s) is limited to the group of diacylperoxides (of Formula II) and/or peroxyesters (of Formula III), comprising optionally, but preferably, at least 0.01 to 1% w/w of one or more substances that have a stabilizing effect on the decomposition rate of a peroxide, which are phlegmatised with an agent of Formula IV.

The peroxide compositions are prepared in a conventional way by mixing the stabiliser (if used), one or more peroxides, and phlegmatising agent in any sequence at temperatures below the SADT of the peroxides. Alternatively, the peroxide is produced in the phlegmatising agent, requiring just the optional mixing in of the stabiliser.

The invention is elucidated by the following examples.

Experimental

The thermal stability was tested using the mini Heat Accumulation Storage Test (m-HAST). In this test, an appropriate sample (50 g) is put into a Dewar vessel (100 mL) and stored at a desired test temperature. During the test, the temperature of the sample is continuously measured. The experiment is stopped after a runaway occurred, or after a specified maximum time (i.e. no runaway).

### EXAMPLE 1 AND COMPARATIVE EXAMPLES A-B

Trigonox® EHPS (di-2-ethylhexyl peroxydicarbonate that is stabilised with tert-butyl hydroperoxide) ex Akzo Nobel was combined with conventional phlegmatiser, i.e. isododecane or cyclohexene (comparative examples A-B), or with a phlegmatiser according to the invention, i.e. 1-octene (example 1).

| Example | A | B | 1 |
|---|---|---|---|
| Peroxydicarbo- | Trigonox ® | Trigonox ® | Trigonox ® |
| nate + | EHPS + 75% | EHPS + 75% | EHPS + 75% |
| amount¹⁾ of | w/w isododecane | w/w cyclohexene | w/w 1-octene |
| phlegmatiser | | | |
| m-HAST at | 15° C. | 15° C. | 15° C. |
| Induction time | 90 | no runaway | no runaway |
| before decom- | | | |
| position (hours) | | | |
| Thermal Explo- | 6 | 6 | 4 |
| sion Vessel | | | |
| (TEV) max. | | | |
| pressure (barg) | | | |
| Dutch Pressure | 3.5 | 3.5 | 3.0 |
| Vessel test | | | |
| Performance in | good | unacceptable | good |
| PVC polymer- | | | |
| isation | | | |
| kinetics | | | |

¹⁾based on the weight of the total composition (% w/w)

### EXAMPLE 2 AND COMPARATIVE EXAMPLES C-D

Unstabilised di-2-ethylhexyl peroxydicarbonate (Trigonox® EHP) ex Akzo Nobel was combined with a conventional phlegmatiser, i.e. isododecane or cyclohexene (comparative examples C-D), or with a phlegmatiser according to the invention, i.e. 1-octene (example 2).

| Example | C | D | 2 |
|---|---|---|---|
| Peroxydi- | Trigonox ® | Trigonox ® | Trigonox ® |
| carbonate + | EHP + 75% | EHP + 75% | EHP + 75% |
| amount¹⁾ of | w/w isododecane | w/w cyclohexene | w/w 1-octene |
| phlegmatiser | | | |
| m-HAST at | 15° C. | 15° C. | 15° C. |
| Induction time | 11 | no runaway | no runaway |
| before decom- | | | |
| position (hours) | | | |
| Thermal Explo- | 5 | 5 | 3 |
| sion Vessel | | | |
| (TEV) max. | | | |
| pressure (barg) | | | |
| Dutch Pressure | 3.5 mm | 3.0 mm | 3.0 mm |
| Vessel test | | | |
| Performance in | good | not acceptable | good |
| PVC polymer- | | | |
| isation kinetics | | | |

¹⁾based on the weight of the total composition (% w/w)

Unexpectedly, the combination of stabilised and unstabilised peroxydicarbonate and the α-olefinic phlegmatising agent 1-octene (Examples 1-2) resulted in compositions having (all compared to similar peroxide compositions wherein conventional phlegmatiser was used (Comparative examples A-B & C-D)):

Higher runaway temperature.

Longer induction time before the decomposition.

Expected lower heat production at or above storage temperatures.

Lower pressure build-up during the decomposition at or above storage temperatures.

Good performance in PVC polymerisation kinetics.

Also, when these peroxide compositions of the present invention were used in a conventional suspension polymerisation of vinyl chloride monomer, it was observed that:

Less volatiles were found in the PVC.

PVC was obtained with unexpected properties regarding morphology, processing and stability.

1. A peroxide composition that comprises:
at least one peroxide of the formula: 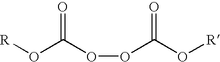
, and optionally at least one peroxide of the formulae: 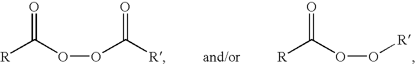
wherein R and R′ are independently selected from branched or non-branched, substituted or unsubstituted alkyl, alkenyl, or cycloalkyl C₁-C₂₀ hydrocarbon moieties,
optionally at least one substance that has a stabilising effect on the decomposition rate of a peroxide, and
at least one phlegmatising agent selected from the group of unsaturated compounds of the general formula R″HC═CHR′″ wherein R″ and R′″ are independently selected from hydrogen and the group consisting of linear or branched, substituted or unsubstituted, saturated or unsaturated C₁-C₁₂ alkane moieties, and where R″ and R′″ may be connected to form a cyclic structure, with the exception of cyclohexene:

2. A peroxide composition that comprises:
at least one peroxide of the formulae: 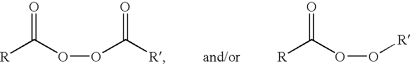
wherein R and R′ are selected from the group of hydrocarbon moieties according to the definition as described in claim 1,
optionally at least one substance that has a stabilising effect on the decomposition rate of a peroxide, and
at least one phlegmatising agent selected from the group of unsaturated compounds of the general formula R″HC═CHR′″ wherein R″ and R′″ are independently selected from hydrogen or the group of alkane moieties according to the definition as described in claim 1.

3. A composition according to claim 1 or 2 wherein the unsaturated phlegmatising agent is selected from the group of 1-hexene, 1-heptene, 1-octene, 1-nonene, 1-decene, 1-undecene, 1-dodecene, cyclooctene, and cyclodecene, or mixtures thereof.

4. A composition according to any one of claims 1 or 2 wherein the unsaturated phlegmatising agent is 1-octene.

5. A composition according to claim 1 wherein the peroxide composition comprises:
from 20 to 95% by weight, based on the weight of the total composition (% w/w), of at least one peroxide of the formula: 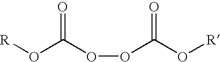
, and optionally at least one peroxide of the formulae: 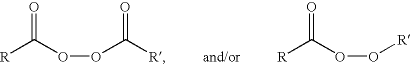
wherein R and R′ are independently selected from the group of hydrocarbon moieties according to the definition as described in claim 1,
from 0 to 1% w/w of at least one substance that has a stabilizing effect on the decomposition rate of a peroxide,
from 90 to 5% w/w of at least one phlegmatising agent selected from the group of unsaturated compounds according to the definition as described in claim 1, said phlegmatising agent preferably being reactive enough to react effectively in the polymerisation process, and from 0 to 50% w/w of at least one optional conventional phlegmatising agent,
up to a total of 100% .

6. A composition according to claim 2 wherein the peroxide composition comprises:
from 20 to 95% by weight, based on the weight of the total composition (% w/w), of at least one peroxide of the formulae: 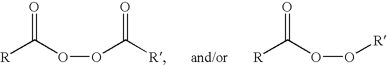
wherein R and R′ are independently selected from the group of hydrocarbon moieties according to the definition as described in claim 1,
optionally from 0.01 to 1% w/w of at least one substance that has a stabilizing effect on the decomposition rate of a peroxide,
from 90 to 5% w/w of at least one phlegmatising agent selected from the group of unsaturated compounds according to the definition as described in claim 1, said phlegmatising agent preferably being reactive enough to react effectively in the polymerisation process, and
from 0 to 50% w/w of at least one optional conventional phlegmatisers, up to a total of 100%.

7. A method to produce a polymer by means of a radical polymerisation process involving the step of using a peroxide composition according to any one of claims 1-2 or 5-6.

8. A method to produce a polymer according to claim 7, wherein the polymerisation conditions are selected such that at least 25% w/w of the reactive phlegmatiser that was used is not extractable from the polymer.

9. A method to safely produce, transport, and otherwise handle a peroxide composition by selecting a phlegmatised, and optionally stabilised composition according to any one of claims 1-2 or 5-6.
